(19) United States
(12) Patent Application Publication
(10) Pub. No.: US 2020/0219599 A1
(43) Pub. Date: Jul. 9, 2020

(54) Systems and Methods of Generating Patient Notes with Inherited Preferences

(71) Applicant: Modernizing Medicine, Inc., Boca Raton, FL (US)

(72) Inventors: Daniel Cane, Boynton Beach, FL (US); Michael Sherling, Boca Raton, FL (US); Adam Gresh, Boynton Beach, FL (US)

(73) Assignee: Modernizing Medicine, Inc., Boca Raton, FL (US)

(21) Appl. No.: 16/825,087

(22) Filed: Mar. 20, 2020

Related U.S. Application Data
(63) Continuation of application No. 15/730,831, filed on Oct. 12, 2017, now abandoned.
Application No. 15/730,831 is a continuation of application No. 14/015,230, filed on Aug. 30, 2013, now abandoned.

(51) Int. Cl. G16H 10/60; G06F 21/62; G16H 10/20

(52) U.S. Cl. CPC G16H 10/60; G16H 40/63; G16H 10/20; G06F 21/6245

(57) ABSTRACT

In part, the invention relates to a computerized system and method of allowing a user to take medical notes for a second person. In one embodiment, the method includes the steps of: logging on to the computer medical records system using a user interface, as a user; accessing a user database by the computer medical records system to determine whether the user has sufficient permissions to inherit the preferences of the second person; if the user has sufficient permission to inherit the preferences of the second person, allowing, by the computer medical records system, the user to inherit the preferences of the second person; and if the user does not have sufficient permission, allowing, by the computer medical records system, the user to inherit the user's own preferences.

### FIELD OF THE INVENTION

The invention relates generally to the field of medical records and more specifically to the recordation and retrieval of patient information by one user on behalf of another.

### BACKGROUND OF THE INVENTION

Studies have shown that physicians spend 45% of their time outside of the examination room. A good deal of that time is spent filling out paperwork, such as patient notes and billing documentation. It takes the average physician two minutes to dictate a note. Even computer-literate clinicians take four minutes to record a note using standard electronic medical records software. Therefore, the act of completing a patient record is time consuming.

Because of these complicated billing requirements, some physicians under-code their billing information. Because these physicians are concerned that mistakes in billing entries might lead to an audit, these physicians will under-code or claim to have done less work than they actually performed to avoid missing something in the required documentation.

Conversely, physicians may mistakenly over-code. This occurs when the physician appropriately codes for a certain level visit based on what has occurred during the visit, but forgets to enter key components in his/her documentation. The Centers for Medicare and Medicaid Services (“CMS”) treats this as an over-code. As far as CMS is concerned, if the documentation is not correct, the examination or procedure did not occur.

Having a physician draft notes and document the diagnosis and related interventions is time consuming, fraught with errors and inefficient. To relieve these issues, a number of systems exist that provide template patient records into which physicians enter data on the computer. In general, these systems barely reduce the time it takes for a physician to enter a note into the medical record of a patient.

One way to increase clinician utilization is to have someone else record the findings of the clinician during the examination. However, because each person's writing style is different, a clinician may still be required to spend an inordinate amount of time editing the notes to correspond to his or her writing style. What is needed is an automated system for generating medical records from minimal input that still records the preferences and style of the clinician in the notes.

The present invention addresses this need and others.

### SUMMARY OF THE INVENTION

In one aspect, the invention relates to a computerized method of allowing a user to take medical notes for a second person. In one embodiment, the method includes the steps of: logging on to the computer medical records system using a user interface, as a user; accessing a user database by the computer medical records system to determine whether the user has sufficient permissions to inherit the preferences of the second person; if the user has sufficient permission to inherit the preferences of the second person, allowing, by the computer medical records system, the user to inherit the preferences of the second person; and if the user does not have sufficient permission, allowing, by the computer medical records system, the user to inherit the user's own preferences.

In another embodiment, the method further includes the step of prompting the user, by the computer medical records system, to determine who the second person is. In yet another embodiment, the method further includes the step of determining, by the computer medical records system, whether the user accepts the preferences of the second person. In still yet another embodiment wherein if the user does not accept the preferences of the second person, the method includes the step of allowing, by the computer medical records system, the user to use the preferences of the user. In one embodiment, the determination of preferences, by the computer medical records system, is done for both read and write preferences.

In another aspect, the invention relates to a medical records system for allowing a user to take medical notes for a second person. In one embodiment, the system includes: a user interface for allowing the user to log on to the computer medical records system; a user data base comprising user permissions and second person preferences; a processor for accessing the user database to determine whether the user has sufficient permissions to inherit the preferences of the second person; if the user has sufficient permission to inherit the preferences of the second person, allowing, by the processor, the user to inherit the preferences of the second person; and if the user does not have sufficient permission, allowing, by the processor, the user to inherit the user's own preferences.

In another embodiment, the medical records system includes a processor to prompt the user using the user interface, to determine who the second person is. In yet another embodiment, the processor determines whether the user accepts the preferences of the second person by reading the input of the user from the user interface. In still yet another embodiment, if the user does not accept the preferences of the second person, allowing, by the processor, the user to use the preferences of the user. In one embodiment, the determination of preferences, by the processor, is done for both read and write preferences.

In another aspect, the invention relates to a medium such as a non-transitory medium or memory that includes one or more executable programs or software modules configured to respond to user inputs when inputting or retrieving patient data via a graphical user interface. The executable programs or software modules can include adaptive algorithms configured to analyze data sets of user preferences and generate user profiles suitable for collecting or retrieving patient information based on user preferences and historic data entry or retrieval using the graphical user interface. In one embodiment, the method includes the steps of providing an input screen on a computer system; inputting data to the computer system; accessing a database on the computer system to obtain patient data in response to data input to the input screen; and tracking impressions of the user by frequency with regard to one or more user selectable preferences.

In another aspect, the invention relates to a medical records system and related methods by which a user electronically records patient data relative to a visual representation of the patient using a graphical user interface. The patient data can include diagnosis data and treatment data. The choices made by the user when electronically recorded and/or when retrieving patient data are stored as part of a historic record for the user in a database. In one embodiment, preferences of the user are determined using one or more software modules and a computing device to correlate the frequency of particular user's selections relative to a patient (or other clinicians or data entry personnel) such as procedures for a particular diagnosis. Patient demographic data can also be used to generate a historical record of the types of patients a given user typically diagnoses or treats or handles billing records for over time. Other insurance and medical record data can be tracked and used to generate user preference models based on the frequency of selection and other inputs received via the graphical user interface over time for different patients, diagnoses, and procedures.

In one embodiment, frequency tracking of inputs via the graphical user interface is implemented as an impression count table in a database. User preferences can also be determined by extracting details such as text description used or units of measure from previous procedures that match the type of procedure currently being input using the graphical user interface. In this way, the software modules can carry forward any customization, input data or other data stored relative to a patient from a previous procedure. In one embodiment, the top ten recommendations by frequency, for a particular diagnosis are displayed using a graphical user interface.

In one embodiment, permissions are set from person to person to regulate how user preferences are adapted and changed. In other words, anywhere a choice can be made relative to the interfaces (which are tracked as part of the adaptive learning algorithm), a user can inherit those choices as part of their preferences. The software modules can characterize a user as trusted or untrusted based on read/write permissions. A user, such as a clinician, can grant another person permission to use their preferences. As the other person makes choices, the system, if it has a permission set to allow it, can weigh the other person's choices and factor them into future preferences for the user. In the second scenario, with an untrusted second user, the permission granted is to use preferences of first user, such as a clinician, but any choices the second person makes are not allowed by the system to change future preferences recorded for first user.

### BRIEF DESCRIPTION OF THE DRAWINGS

The objects and features of the invention can be understood more completely by referring to the drawings described below and the accompanying descriptions.

FIG. 1 is a highly generalized schematic diagram of an overview of an embodiment of a system constructed in accordance with the invention;

FIG. 2 is a highly generalized schematic block diagram of an embodiment of a system with various software modules constructed in accordance with the invention;

FIGS. 3A and 3B are embodiments of a data structure utilized in an embodiment of the system;

FIGS. 4A and 4B are flowcharts of an embodiment of the system operated in accordance with the invention;

FIGS. 5A-5G are embodiments of graphical user interfaces as seen by a user in performing in accordance with an embodiment of the invention; and

FIG. 6 is an embodiment of an impression count table that may be used in a database for user preference tracking and profile generation in accordance with an embodiment of the invention.

### DETAILED DESCRIPTION

In part, one embodiment of the invention relates to systems and methods that use a computing device such as a tablet, smart phone, desktop computer, or other device to streamline the process of evaluating a patient and retrieving information relating thereto. A first user, such as a clinician, and a second user, such as an assistant or insurance data entry specialist, can work together using the system and user interface, permission, and adaptive features described herein to reduce the time required to complete patient notes while still generating patient notes consistent with the clinician's preferences. A user of the methods, systems and other technologies described herein interfaces with a device that includes a browser or one or more graphic user interfaces configured to capture and/or retrieve patient data. Visual representations of the patient are typically part of one or more of the interface screens with respect to which data is collected, displayed and stored.

This data capture and retrieval is often in the context of performing a patient exam or diagnosis in which notes relating to the patient are generated by a clinician or someone working with the clinician. In a given medical practice, various users with their own styles and preferences handle various aspects of the diagnosis, billing, note taking and other data entry and retrieval functions associated with a practice.

Upon accessing the system with using a graphical user interface, such as a touch screen interface on a tablet or smart phone, the clinician or a second person assisting the clinician can obtain from the database selectable lists of patients, and once selected, a user interface configured to anticipate the needs of the user based on historic user preferences even if the data is being entered by the second user and not the clinician.

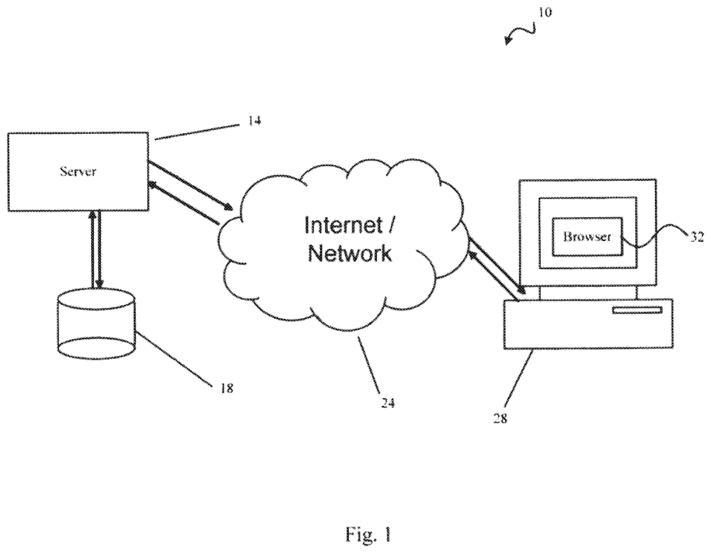

In brief overview and referring to FIG. 1, a system 10 constructed in accordance with an embodiment of the invention is shown. The system 10 includes a server 14 having access to a database 18. The server 14 is in communication, through a network 24, such as the Internet, with a client 28. In one embodiment, the client 28 has a browser 32 such as a touch screen interface on a table or a web browser for a remote application. Other methods for communicating over the network are contemplated. The client 28 can be implemented in software, which is preferred, or hardware.

In various embodiments, the client 28 can be a mobile device including, without limitation, desktop computers, laptop computers, network computers, tablets, smart phones. A clinician, such as a physician, physician's assistant, or medical assistant, using the system 10, can communicate with the server 14 using the client 28. In one embodiment, the clinician is examining a patient and providing comments to a second user who records and retrieves data relating to the patient, but the preference profile that governs the data recordation is that of the clinician.

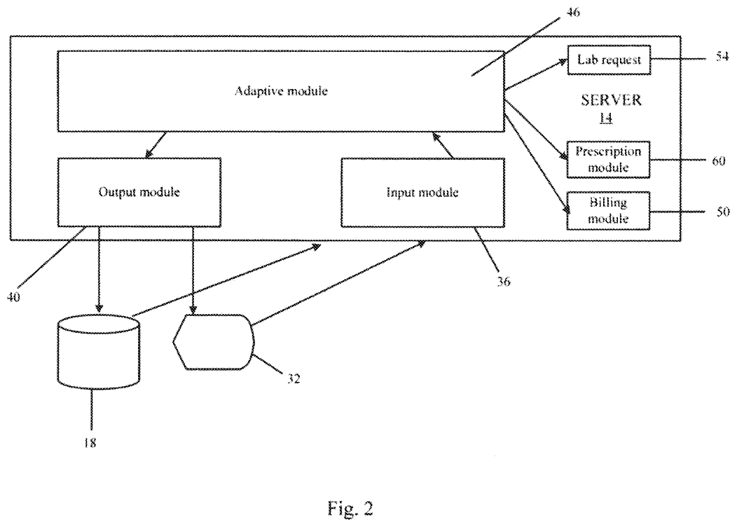

Referring now to FIG. 2, in one embodiment, the server 14 generally includes multiple modules; these modules can include, without limitation, an input module 36 for obtaining input data both from the database 18 and the browser 32; an output module 40 for writing data to both the database 18 and the browser 32; and an adaptive notes generation module 46 in communication with the input module 36 and the output module 44. The adaptive notes generation module 46 uses the data input from the browser 32 to obtain additional data from the database 18 not only to construct and populate an output screen for display on the browser 32 but also to create physician notes to be entered as part of the patient record in the database 18. The adaptive notes generation module 46 can access and update preference records based on set permission levels that are stored in database 18 or in another memory store.

In other embodiments, the server 14 includes other modules in communication with the adaptive note generation module 46 such as, but not limited to: a billing module 50, a laboratory request module 54, and a prescription module 60. The adaptive note generation module 46 provides the billing codes to the billing module 50 and ensures that all the proper documentation is completed. If the adaptive note generation module 46 detects missing information required for payment, the clinician is notified. The adaptive note generation module 46 provides the laboratory request module 54 with requests for laboratory tests and surgical test procedures to be performed on the patient. The adaptive note generation module provides the prescription module 60 with the medications prescribed for the patient. In turn, the prescription module 60 transmits prescription requests to the pharmacy (not shown). Other data collection, processing and transforming modules can also be used in various embodiments.

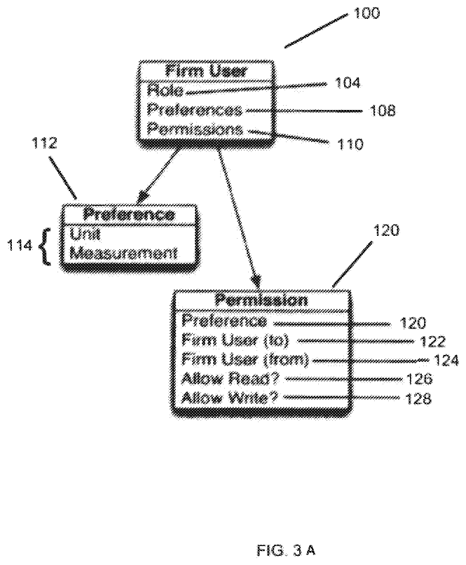

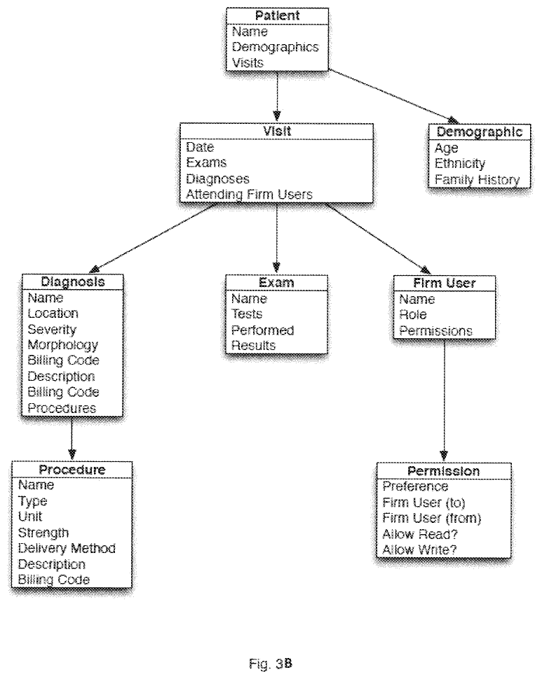

Referring to FIG. 3A, an example of an embodiment of a data structure for use with an embodiment of the invention is shown. In the embodiment shown, a user object 100 (also referred to as a firm user for a particular practice group or medical care provider) exists for each user of the system. Associated with the user object 100 is the user's role 104, (for example physician, nurse, physician's assistant, therapist, medical assistant, etc.); a pointer 108 to the preferences object 112 of the user; and a pointer 110 to the permissions object 120 of permissions granted by the user. As the user documents patient treatments, a history of choices is recorded in the database 18.

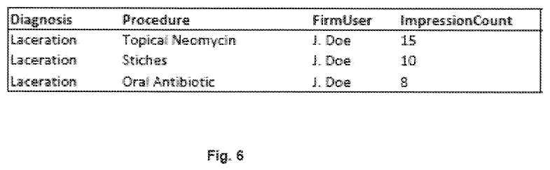

In one embodiment, the preferences 112 of the user are determined by correlating the frequency of particular procedures to a particular diagnosis and, in another embodiment, the preferences 112 of the user are determined by correlating the frequency of particular procedures to patient demographics. In some embodiments, this can be implemented as an impression count table in the database, to increase efficiency (FIG. 6). The adaptive software module can also evaluate the previous procedures of this type, to present any customizations or text or other input data from a previous procedure.

Referring to FIG. 3B, an example of an embodiment of additional data structures and relationship between such structures for use with an embodiment of the invention is shown. The relations between data stored in an embodiment of the database can be view relative to a patient record for the patient being examined. In one embodiment, the patient record is relationally tied to a visit record and a demographic record. For a given visit, the data stored can be linked to a firm user/user object 100 in database 18. The visit record for the patient can also be linked to an exam record for the patient and a diagnosis resulting from the exam. The diagnosis record can be linked to a procedure record. The firm user record is also linked to a permission record as shown in one embodiment. Various records and relationships between them as shown in FIG. 3 can be frequency counted, and each element in a record and among records (such as the location element and bill code element for the diagnosis record and the name of the procedure element and the billing code element for the procedure record) can be correlated with other records and elements using the adaptive module. This allows the system to anticipate how a given firm user will want to use the user interface to capture and retrieve data such as by prepopulating the interface with the diagnosis and procedure billing codes based on the diagnosis element entered.

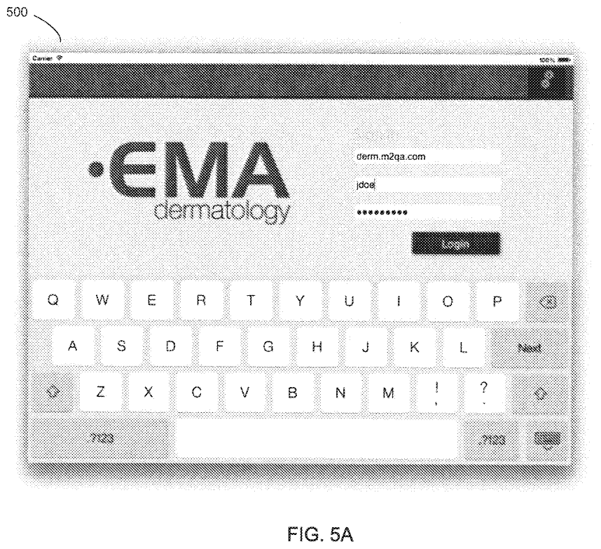

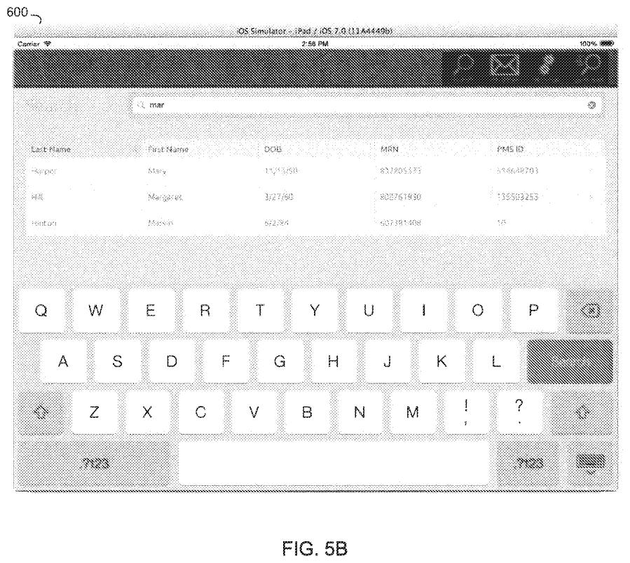

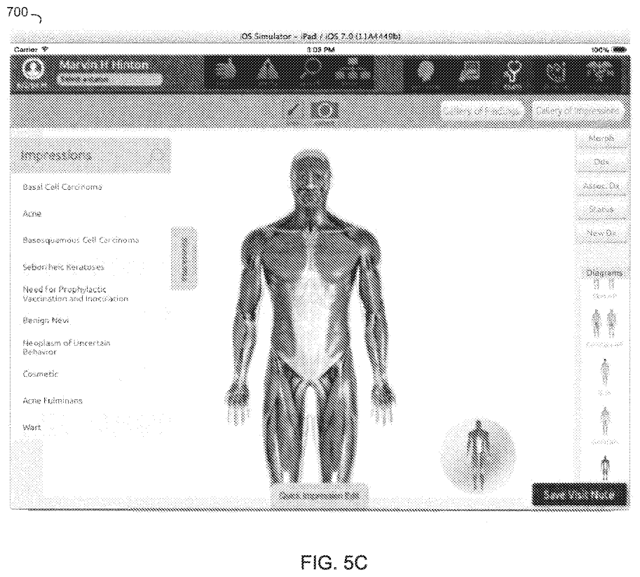

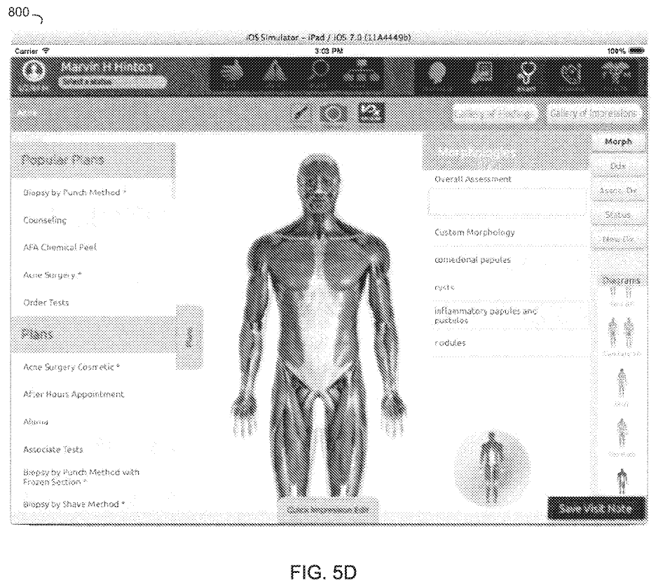

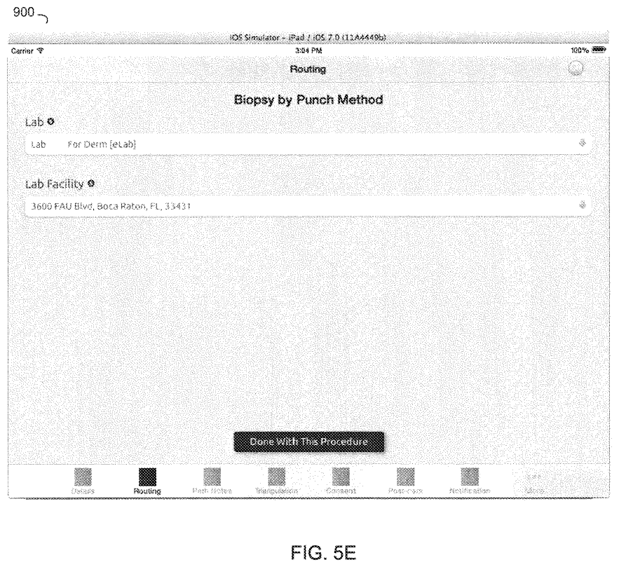

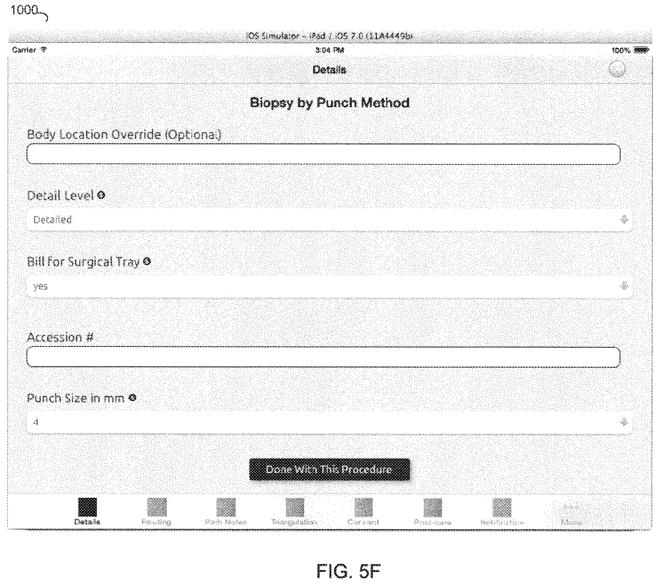

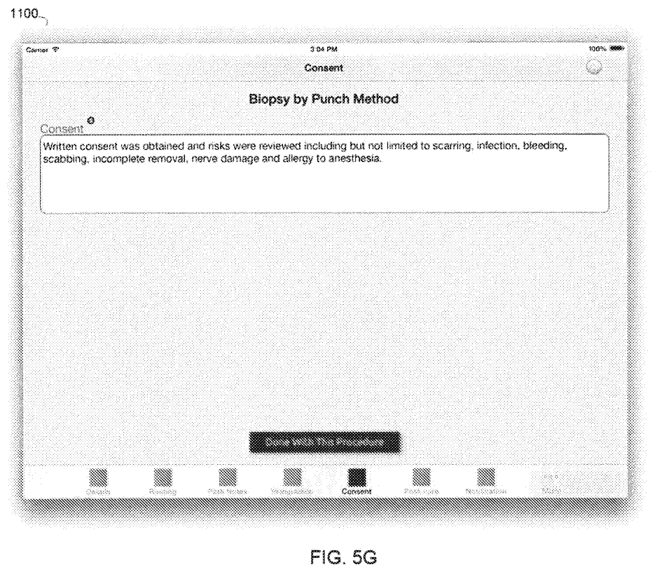

Frequency, counts, and correlations between data entered via the graphical user interface (such as shown in FIG. 5A to G) are used to generate a database of user preferences (such as the preference elements in the firm user record) which can be ranked, scored, or otherwise correlated for display during procedures in the future in anticipation of user needs. Prepopulated lists, reordered menus, and navigating to areas of the form that are focused upon by the user, can all be generated based upon historic user preferences stored in the database and processed using one or more software modules, such as the adaptive software module.

Preferences 114 within the preferences object 112 are the preferences of the user for writing patient notes. These preferences vary from the simple, such as what units of measurement the user prefers (English, metric, decimal, etc), to what the preferred form the note should take for a given diagnosis. This complex preference is determined by the adaptive notes generation module 46 (FIG. 2) based on the user's prior notes and is displayed as options to the user.

In some embodiments, customized text data or other input data from prior notes is presented to the user or, in other embodiments, the top ten recommendations by frequency for the particular diagnosis are presented to the user. By way of an example only, a notation in the system stating that a patient has a 1 inch deep cut on the inside of the patient's right lower arm requiring stitches might be described in a note generated by the system as “Patient presents with a 2.5 cm laceration on the ventral surface of the patient's right forearm requiring sutures. Patient is prescribed topical neomycin for application three times daily.” This note would be generated because the physician's preferences indicate that the physician preferred to use metric units.

Also, once it is determined that the physician intends to treat with an antibiotic, a list of antibiotics are presented to the physician based on past popular treatments. The treatments presented are based on past preferences—the physician in this example frequently used topical neomycin when prescribing antibiotics for a sutured wound. Treatments may also be presented based on the top ten recommendations by frequency for the treatment of sutured wounds. Once the treatment is chosen, details and text or other data related to this treatment are generated and populated based on this physician's past decisions.

The permissions object 120 includes a table of to whom the user gives permission 122; from whom the user obtains permission 124, and whether the people to whom and from whom the user gives and obtains permission have given read 126 and write 128 privileges. Permissions may be global from person to person. In other words, anywhere a choice can be made (which may be tracked as part of the adaptive learning algorithm), a user may inherit those choices. There may be different read/write permissions scenarios, for example, a trusted second user or scribe, and an un-trusted second user or scribe. In the case of the trusted scribe, permission may be granted to use preferences. As the trusted scribe make choices, the user, such as the diagnosis clinician, granting permission may trust the trusted scribe enough to have those choices factor into future preferences of the clinician. In the case of the un-trusted scribe, permission may be granted to use preferences, but any choices the un-trusted scribe makes should not factor into future preferences of the clinician.

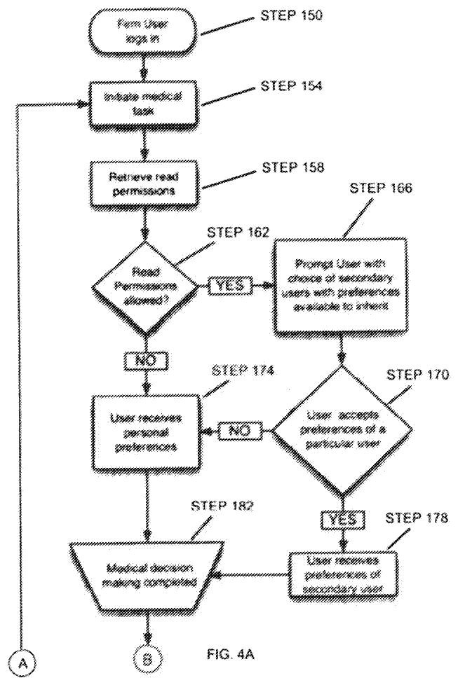

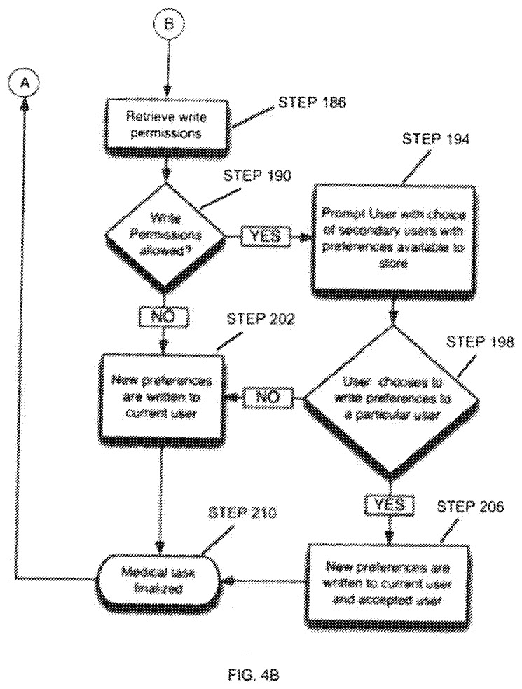

Referring next to FIGS. 4A and 4B, to use the system a user such as a medical assistant logs into the system (Step 150) as himself or herself. FIG. 5A shows an exemplary login relative to a dermatology practice which is included for context as a non-limiting example. The user then initiates the task (Step 154) such as requesting that the system provide an examination form for the patient to be examined. The system determines whether the user has sufficient permission (Step 158) to access or read the list of clinicians, etc. for whom the user will be generating notes.

If the user has the requisite read permission (Step 162), the system prompts the user to select the name of the clinician whose preferences the user will be accepting (Step 166) and asks the user to accept the preferences (Step 170). If the user does not accept the preferences or if the user did not have read permission (Step 162), the user receives only his or her personal preferences (Step 174). If the user accepts the preferences of the clinician, then the user receives the preferences of the clinician (Step 178) and then utilizes the preferences in medical decision-making and documentation. (Step 182).

As a user actively documents the medical decision making using the software-based interface on a tablet or other computing devices, the choices the user makes factor into the user's preferences and therefore factor into the options this user will receive in the future (subject to the trusted or untrusted permission level). Next, the system proceeds to determine whether (Step 186) the user has write permissions, and if so (step 190), again prompts (Step 194) the user to determine to which clinicians the preferences should be written.

At this time, the user may accept to write the preferences (Step 198) to a clinician (Step 206), in which case those preferences are written to the user and to the chosen clinician (Step 206). If the user does not accept to write the preferences to the clinician (Step 202) or does not have write permissions (Step 190), then new preferences are written to the user (Step 202). In either case, the notes are written to the patient's record (Step 210).

FIG. 5A-5G are graphical user interfaces (GUI) as seen by the user in accordance with the invention. These are shown relative to dermatological treatment and diagnosis patient notes, but are generalizable to any treatment, procedure, or medical specialty or general practice. These can be used to access the system and document a patient exam or retrieve patient data. Referring now to FIG. 5A, an embodiment of a logon screen 500 in accordance with invention is shown. Logon screen 500 may, in part, allow a user to logon to the medical records system.

Referring now to FIG. 5B, a patient search GUI 600 is shown. Patient search GUI 600 may, in part, allow a user to search for a specific patient. Patient search GUI 600 may, in response to search characters being entered by the user, show a number of patients from which a user may choose. FIG. 5B shows one embodiment of a list of patients displayed in the present invention.

Referring now to FIG. 5C, an impressions GUI 700 is shown. Impressions GUI 700 may display one or more choices of diagnosis, which may be ordered based on historical preference. Given a clinician that routinely deals with one type of demographic population, this list can be ordered based on the impressions they typically encounter relative to that demographic population.

Referring now to FIG. 5D, a preferred plans GUI 800 is shown. Preferred plans GUI 800 may display one or more treatment plans or procedures, which may be based on past preferences in similar diagnoses. Given a clinician's preferences for a procedure based on a given diagnosis, the options shown in FIG. 5D change accordingly in light of the diagnosis being entered into the user interface of FIG. 5C. As example, the second person taking notes using the interface can select biopsy by punch in response to the clinician's diagnosis.

Referring now to FIG. 5E, lab choice GUI 900 is shown. Lab choice GUI 900 may include a circled S or other indicator, which may denote a sticky or priority value, which may be based on past preferences. The sticky or priority values are displayed first based on historic selections of the firm user. Each of the Lab name selectable field and Lab Facility selectable filed are marked with circled S indicator as default values for the user. As shown in FIG. 5E, the choice of a lab to analyze biopsy samples may be selected by the user.

Referring now to FIG. 5F, detail options GUI 1000 is shown. Detail options GUI 1000 may show stickyDetailOptions, which may include a circled S or other indicator. The circled S or other indicator may denote other prioritized or sticky values, where past preferences for treatment plan details may be carried forward. The system generates prepopulated default or sticky values, based on prior data entry and user preferences, to speed patient note taking.

Referring now to FIG. 5G, consent GUI 1100 is shown. Consent GUI 1100 may show consent information in the form of text or other patient record information, where a circled S or other indicator may denote a sticky value for text or other input data concerning the consents that have been signed by a patient.

FIG. 6 shows frequency tracking relative to Diagnosis, Procedure, User, and Impression Count represented by a database table. Typically, this data is collected using the graphical user interface over time to build a historic record for user tracking and correlation processes. These processes can be implemented using the adaptive software module and or other software modules.

Throughout the application, where compositions are described as having, including, or comprising specific components, or where processes are described as having, including, or comprising specific process steps, it is contemplated that compositions of the present teachings also consist essentially of, or consist of, the recited components, and that the processes of the present teachings also consist essentially of, or consist of, the recited process steps.

In the application, where an element or component is said to be included in and/or selected from a list of recited elements or components, it should be understood that the element or component can be any one of the recited elements or components and can be selected from a group consisting of two or more of the recited elements or components. Further, it should be understood that elements and/or features of a composition, an apparatus, or a method described herein can be combined in a variety of ways without departing from the spirit and scope of the present teachings, whether explicit or implicit herein.

It should be understood that the order of steps or order for performing certain actions is immaterial so long as the present teachings remain operable. Moreover, two or more steps or actions may be conducted simultaneously.

Computer program logic implementing all or part of the functionality previously described herein may be embodied in various forms, including, but in no way limited to, a source code form, a computer executable form, and various intermediate forms (e.g., forms generated by an assembler, compiler, linker, or locator). Source code may include a series of computer program instructions implemented in any of various programming languages (e.g., an object code, an assembly language, or a high-level language such as JAVA, Javascript, DHTML, AJAX, CSS, XML, SQL, HTML, Fortran, C, Objective-C, or C++) for use with various operating systems or operating environments. The source code may define and use various data structures and communication messages. The source code may be in a computer executable form (e.g., via an interpreter), or the source code may be converted (e.g., via a translator, assembler, or compiler) into a computer executable form.

The examples presented herein are intended to illustrate potential and specific implementations of the invention. It can be appreciated that the examples are intended primarily for purposes of illustration of the invention for those skilled in the art. There may be variations to these diagrams or the operations described herein without departing from the spirit of the invention. For instance, in certain cases, method steps or operations may be performed or executed in differing order, or operations may be added, deleted or modified.

Variations, modification, and other implementations of what is described herein will occur to those of ordinary skill in the art without departing from the spirit and scope of the invention as claimed. Accordingly, the invention is to be defined not by the preceding illustrative description, but instead by the spirit and scope of the following claims.

1. A computerized method of allowing a user to take medical notes for a second person comprising the steps of:
tracking permissions associated with medical notes using an adaptive notes generation module;
logging on to a computer medical records system using a user interface, as a user;
accessing a user database by the computer medical records system to determine whether the user has sufficient permissions to inherit the preferences of the second person;
if the user has sufficient permission to inherit the preferences of the second person, allowing, by the computer medical records system, the user to inherit the preferences of the second person;
if the user does not have sufficient permission, allowing, by the computer medical records system, the user to inherit the user's own preferences; and
creating medical notes, using the adaptive notes generation module, using either the preferences of the second person or the user's own preferences.

2. The method of claim 1 further comprising the step of prompting the user, by the computer medical records system to determine identity of the second person.

3. The method of claim 2 further comprising the step of determining, by the computer medical records system, whether the user accepts the preferences of the second person.

4. The method of claim 3 wherein if the user does not accept the preferences of the second person, allowing, by the computer medical records system, the user to use the preferences of the user.

5. The method of claim 1 wherein the determination of preferences, by the computer medical records system, is performed for both read and write preferences.

6. A medical records system for allowing a user to take medical notes for a second person comprising:
a graphical user interface for allowing the user to log on to the computer medical records system;
a user database comprising user permissions and second person preferences;
a computer processor for accessing the user database to determine whether the user has sufficient permissions to inherit the preferences of the second person;
if the user has sufficient permission to inherit the preferences of the second person, allowing, by the processor, the user to inherit the preferences of the second person; and
if the user does not have sufficient permission allowing, by the processor, the user to inherit the user's own preferences.

7. The medical records system of claim 6 where the processor prompts the user using the user interface, to determine identity of the second person

8. The medical records system of claim 7 wherein the processor determines whether the user accepts the preferences of the second person, by reading the input of the user from the user interface.

9. The medical records system of claim 8 wherein if the user does not accept the preferences of the second person, allowing, by the processor, the user to use the preferences of the user.

10. The medical records system of claim 6 wherein the determination of preferences, by the processor, is performed for both read and write preferences.

11. A method of recording patient notes by using a graphical user interface comprising:
providing an input screen on a computing device to a user in response to a login identifying the user;
accessing a preference record for the user in response to the identity of the user;
displaying fields and a visual representation of a patient for data entry;
displaying data input preferences determined based upon impression data obtained with regard to the user over time and the preference record; and
updating one or more of the impression data and the preference record in response to selections for patient notes.

12. The method of claim 11 further comprising storing a frequency count of impressions on a per user basis and generating a set of user preferences for displaying using the graphical user interface in response to the frequency count.
